(19) United States
(12) Patent Application Publication
(10) Pub. No.: US 2003/0138423 A1
(43) Pub. Date: Jul. 24, 2003

(54) Method of treating inflammation with HIV-1 protease inhibitors and their derivatives

(75) Inventors: Moshe Arditi, Encino, CA (US); Ozlem Equils, Sherman Oaks, CA (US)

Correspondence Address: Pillsbury Winthrop LLP, Intellectual Property Group, Suite 2800, 725 South Figueroa Street, Los Angeles, CA, 90017-5406, US

(73) Assignee: CEDARS-SINAI MEDICAL CENTER

(21) Appl. No.: 10/306,000

(22) Filed: Nov. 27, 2002

Related U.S. Application Data
(60) Provisional application No. 60/340,507, filed on Dec. 14, 2001.

(51) Int. Cl. A61K 39/395; A61K 31/415; A61K 31/551

(52) U.S. Cl. 424/145.1; 514/220; 514/406

(57) ABSTRACT

Disclosed herein are methods of treating inflammation by administering to a patient an HIV-protease inhibitor. While protease inhibitors are known for their capacity to prevent or significantly hinder the cleavage of proteins that would otherwise become smaller viral particles, certain of these inhibitors may also be employed in the treatment of inflammation and diseases in which an inflammatory response is evident. Further methods are directed to the treatment of disease conditions in which it may be advantageous to down-modulate NF-κB cell signal transduction.

### RELATED APPLICATIONS

This application claims the benefit of priority under 35 U.S.C. § 119 of provisional U.S. application serial No. 60/340,507, filed Dec. 14, 2001, the contents of which are hereby incorporated by reference.

### FIELD OF THE INVENTION

This invention relates to methods of affecting the immune response with protease inhibitors. More particularly, the invention relates to methods of treating inflammation with HIV-1 protease inhibitors, their analogs, and derivatives.

### BACKGROUND OF THE INVENTION

In order to produce new infectious virus particles, Human Immunodeficiency Virus-1 (HIV-1) employs proteases to cleave newly produced virus proteins into smaller segments. Based in part on this discovery, protease inhibitors were developed to block the function of these proteases. The use of protease inhibitors in the treatment of viral infectious diseases is therefore known; the inhibitors are commonly used, for example, in the treatment of the Human Immunodeficiency Virus (HIV) and Acquired Immune Deficiency Syndrome (AIDS). In fact, protease inhibitors are viewed as the most potent class of antiviral (and antiretroviral) drugs, yet conditions other than HIV infection are not currently treated with protease inhibitors; known studies and reported efficacy relate almost exclusively to their use in the prevention and treatment of HIV infections.

A number of protease inhibitors that are indicated for use in the treatment of HIV-1 and AIDS are presently commercially available. By way of example, these HIV-1 protease inhibitors include amprenavir (available from GlaxoSmithKline, Research Triangle Park, N.C., under the tradename AGENERASE®), indinavir (available from Merck & Co., Inc., West Point, Pa., under the tradename CRIXIVAN®), saquinavir (available from Roche Pharmaceuticals, Nutley, N.J., hereinafter “Roche,” under the tradename FORTOVASE®), saquinavir mesylate (available from Roche under the tradename INVIRASE®), ritonavir (available from Abbott Laboratories, Inc., North Chicago, Ill., hereinafter “Abbott,” under the tradename NORVIR®), lopinavir/ritonavir (available from Abbott under the tradename KALETRA®), and nelfinavir mesylate (available from Agouron Pharmaceuticals, Inc., La Jolla, Calif., under the tradename VIRACEPT®). These protease inhibitors are generally available in an oral formulation, either in capsule or solution form; however various reports in the literature indicate the potential for parenteral formulations of other HIV-1 protease inhibitors that are not presently in clinical use for the treatment of HIV-1 infection. The presently available HIV-1 protease inhibitors are known to decrease the replication of the HIV virus in humans, and are therefore indicated for the treatment of conditions for which decreasing this replication has a beneficial effect; namely HIV and AIDS.

HIV-1 protease inhibitors have shown few therapeutic qualities beyond their use in the treatment of HIV and AIDS. In mice infected with a lymphocytic choriomeningitis virus (LCMV), ritonavir was shown to selectively inhibit the chymotrypsin-like activity of the 20S proteasome, thereby blocking the presentation of antigen to cytotoxic T lymphocytes (CTLs), yet LCMV replication was not affected. The chymotrypsin-like activity of the 20S proteasome is responsible for the digestion of large hydrophobic residues into smaller peptides that, in turn, are delivered to the cell surface for Major Histocompatibility Complex (MHC)-class I antigen presentation. It was therefore postulated that the immune response may be moderated by protease inhibitors, by blocking the presentation of antigen to CTLs. However, other HIV-1 protease inhibitors were not as effective; saquinavir inhibited chymotrypsin-like activity to a significantly lesser degree, while nelfinavir and indinavir exhibited no inhibition at all. Andre et al., “An inhibitor of HIV-1 protease modulates proteasome activity, antigen presentation, and T cell responses,” Proc. Natl. Acad. Sci. USA 95:13120-13124 (1998).

Further kinetic analysis of proteasome activity modulation by ritonavir indicated that the protease inhibitor protected the MB-1 (X) and/or LMP7 proteasome subunits from covalent active site modification with the vinyl sulfone inhibitor 4-hydroxy-3-iodo-2-nitrophenyl-leucinyl-leucinyl-leucine vinyl sulfone (¹²⁵I-NLVS). This suggested that these subunits are prime targets for competitive inhibition by rintonavir, confirming that this protease inhibitor may modulate antigen processing. Schmidtke et al., “How an Inhibitor of the HIV-1 Protease Modulates Proteasome Activity,” J. Biol. Chem. 274(50):35734-35740 (1999).

Although some protease inhibitors are known to have anti-inflammatory properties, none of these are HIV-1 protease inhibitors. For example, U.S. Pat. No. 4,871,727 describes the anti-inflammatory and anti-degenerative properties of a variety of natural compounds isolated from the L-681,512 microorganism. Although their potential use in therapeutic pharmaceutical compositions is discussed, these compounds include only non-HIV-1 protease inhibitors.

Human cells possess protease machinery that plays a significant role in cellular immune response and the production, regulation, and degradation of immune biochemicals, such as cytokines and NF-κB. Cytokines aid the body in combating infection, but, when produced in excessive quantities, can cause tissue destruction and multiple organ failure, as well as septic shock. Similarly, NF-κB is a component of the cell signaling pathway moderating the immune and inflammatory responses, developmental processes, cellular growth, and apoptosis. NF-κB is also active in a number of disease states, including sepsis, severe sepsis, cancer, arthritis, inflammation, asthma, neurodegenerative diseases, and heart disease.

In the HIV context, NF-κB is a key transcription factor that binds to a promoter region of an inflammatory cytokine, causing expression thereof. Cytokines and chemokines then influence the differentiation of CD4+ T cells into Th1 and Th2 phenotypes with predominant CCR5 and CXCR4 expression patterns, respectively. Th1/Th2 predominance and the level of CXCR4 and CCR5 expression may then influence HIV entry and infection of cells. However, bacterial and mycobacterial antigen-induced NF-κB activation also plays a significant role in inflammatory conditions, such as sepsis, severe sepsis, arthritis, and other inflammatory disease conditions.

NF-κB is regulated by interaction with inhibitory IκB proteins, such as IκB-α. A variety of such proteins are known, each having a different binding affinity for NF-κB. These proteins are regulated differently and expressed in a tissue-specific manner. IκB proteins and NF-κB generally reside in the cell cytoplasm, bound to one another in a latent, inactive complex until an IκB kinase is triggered. Many cell signaling pathways may activate an IκB kinase; the enzyme responsible for phosphorylating the IκB protein, leading to its separation from the NF-κB complex and subsequent digestion via proteolysis with an appropriate proteasome. For instance, lipopolysaccharide (LPS), a major component of the outer surface of gram negative bacteria, is known to trigger such a signaling pathway. Once separated from the IκB protein, NF-κB generally translocates to the cell nucleus where it binds to cognate binding sites on DNA and initiates the transcription of components of that further, for example, the inflammatory response.

Sepsis generally refers to a severe infection, either local or bacteremic, which is accompanied by systemic manifestations of inflammation. It is a physiologic response to toxins introduced into the body, and is often caused by the surgical manipulation of body tissue, such as by introduction of a catheter or routine dental work. Sepsis is particularly harmful for those patients whose immune systems have been compromised by an underlying disease, chemotherapy, or in settings of malnutrition. Primary infection is generally observed in the lungs, in the genito-urinary or gastrointestinal tract, or in soft tissues. Severe sepsis (commonly referred to as “septic shock” or “toxic shock”) is often caused by hospital-acquired bacterial infections, and is most frequently observed in immunocompromised patients and the geriatric population.

Sepsis and severe sepsis are responsible for 215,000 deaths each year in the United States, alone. Owing almost entirely to the lack of an effective treatment, the mortality rate of severe sepsis ranges from 28% to 50%. The administration of antibiotics, drainage of large abscesses, and removal of necrotic tissue accompanied with other supportive care is often insufficient to treat the disease, though these treatments are a nearly exhaustive recital of the few options presently available. Recently, however, the Food and Drug Administration approved the use of Activated Protein C for the treatment of severe sepsis, the first pharmaceutical to be approved for such treatment.

Inflammatory responses are associated with diseases other than sepsis and severe sepsis. Rheumatoid arthritis, for example, is characterized in part by an immune response in multiple joints resulting in moderate to severe pain and stiffness limiting a patient's range of movement of the affected joint. While rheumatoid arthritis may be treated with, for example, nonsteroidal anti-inflammatory drugs (NSAIDs) such as aspirin, this form of treatment does not alter the long-term course of the disease, and frequently has unwanted side-effects, such as irritation of the gastrointestinal tract.

Inflammation accompanies numerous other disease conditions, as well. Along with all other forms of arthritis (arthritis, by definition, including not only joint inflammation, but also the inflammation of other bodily connective tissue, such as muscle, tendon, ligaments, and the protective coating of internal organs), inflammation is a symptom of inflammatory bowel disease, ulcerative colitis, and Crohn's Disease. A variety of treatment options may be available for these disease conditions, yet many of those options are subject to the same limitations as those described above for rheumatoid arthritis.

### SUMMARY OF THE INVENTION

It is an object of an embodiment of the present invention to provide methods for treating inflammation or an immune response with protease inhibitors. These methods permit one to treat any disease in which inhibiting inflammation would have a beneficial effect, such as sepsis or severe sepsis, arthritis, inflammatory myocarditis, glomerulonephritis, inflammatory conditions of the gastrointestinal tract, such as inflammatory bowel disease, ulcerative colitis, and Crohn's Disease, and inflammatory conditions of the central nervous system (CNS). HIV-1 protease inhibitors, their analogs, and their derivatives may be used in accordance with the methods of the present invention.

Other features and advantages of the invention will become apparent from the following detailed description, which illustrates, by way of example, various embodiments of the present invention.

### BRIEF DECRIPTION OF THE DRAWINGS

FIG. 1 is a graphical representation of an effect of a protease inhibitor of the present invention on LPS-induced cell activation in human dermal microvessel endothelial cells (“HMEC”). LPS activation was measured in terms of percent NF-κB luciferase activity; luciferase being included in a plasmid joined to NF-κB such that greater bioluminescent detection of luciferase corresponded to greater NF-κB activity. Nelfinavir, a protease inhibitor of the present invention, blocked LPS-induced NF-κB activation in a dose-dependent manner (i.e., significantly greater blocking of NF-κB activation at each of 1 μg/ml, 3 μg/ml, and 6 μg/ml concentrations of nelfinavir, respectively) without inducing apoptosis or death of the cells.

FIG. 2 is a graphical representation of an effect of a protease inhibitor of the present invention on LPS activation in HMEC. LPS activation was measured in terms of percent Human Immunodeficiency Virus Long Terminal Repeat (“HIV-LTR”) luciferase activity; luciferase being included in a plasmid joined to HIV-LTR such that greater bioluminescent detection of luciferase corresponded to greater HIV-LTR activity. Nelfinavir blocked LPS-induced HIV-LTR activation in a dose-dependent manner (i.e., significantly greater blocking of HIV-LTR activation at each of 1 μg/ml, 3 μg/ml, and 6 μg/ml concentrations of nelfinavir, respectively) without inducing apoptosis of the cells.

FIG. 3 is a comparative graphical representation of an effect of several protease inhibitors of the present invention on NF-κB activation in HMEC. LPS-induced cell activation was measured in terms of percent NF-κB luciferase activity, as described previously in FIG. 1. Nelfinavir blocked LPS-induced NF-κB activation in a dose-dependent manner (i.e., significantly greater blocking of NF-κB activation at each of 1 μg/ml, 3 μg/ml, and 6 μg/ml concentrations of nelfinavir, respectively) without inducing apoptosis of the cells. Saquinavir blocked LPS-induced NF-κB activation, as did ritonavir. Indinavir also blocked LPS-induced NF-κB activation, but to a lesser degree.

FIG. 4 is a graphical representation of an effect of a protease inhibitor of the present invention on microbial antigens other than LPS in HMEC. Activation of NF-κB Staphylococcus epidermidis phenol soluble modulin (“PSM”) and soluble Mycobacterium tuberculosis factor (“STF”) were measured in terms of percent NF-κB luciferase activity, as described previously in FIG. 1. Nelfinavir blocked NF-κB activation in both PSM and STF.

FIG. 5 depicts the results of a Western blot analysis indicating an effect of a protease inhibitor of the present invention on LPS-induced IκB-α degradation at 30 minutes and at 90 minutes. At 30 minutes, HMEC pretreated with nelfinavir exhibited markedly less LPS-induced IκB-α degradation than HMEC not treated with a protease inhibitor of the present invention.

FIG. 6 is a graphical representation of a time response of protease inhibitor pretreatment induced inhibition of LPS-activation of HIV-LTR luciferase in HMEC with a protease inhibitor of the present invention. LPS activation was measured in terms of percent HIV-LTR luciferase activity, as described previously in FIG. 2. Nelfinavir blocked LPS-induced HIV-LTR activation in a dose-dependent manner (i.e., significantly greater blocking of HIV-LTR activation at each of 0.5 μg/ml, 2 μg/ml, and 4 μg/ml concentrations of nelfinavir, respectively); nelfinavir pre-treatment at 30 minutes, 60 minutes, and 90 minutes before exposure to LPS had similar effects to suppress LPS-induced HIV-LTR activation.

FIG. 7 is a graphical representation of an effect of a protease inhibitor of the present invention on lactate dehydrogenase (“LDH”) release in HMEC. LDH is released from the cells upon cell death. Pretreatment of the cells with various concentrations of nelfinavir prior to LPS stimulation did not induce cell death.

FIG. 8 is a comparative graphical representation of an effect of several protease inhibitors of the present invention on Interleukin 6 (“IL-6”) luciferase activation in HMEC. Nelfinavir, saquinavir, indinavir, and ritonavir each blocked LPS-induced IL-6 luciferase activation.

FIG. 9 is a comparative graphical representation of an effect of several protease inhibitors of the present invention on LPS-induced Tumor Necrosis Factor-α (“TNF-α”) production in THP-LTR-Luc cells. Saquinavir and indinavir each blocked TNF-α production, and ritonavir and nelfinavir each blocked production to a lesser extent.

### DETAILED DESCRIPTION OF THE INVENTION

The present invention is based on the surprising discovery that HIV-1 protease inhibitors affect the NF-κB signaling pathway by down-modulating the enzymatic digestion of an IκB protein/NF-κB complex into its constituent parts. Such down-modulation may substantially reduce the amount of free NF-κB that translocates from cell cytoplasm to the cell nucleus in response to a cell signal that would otherwise cause NF-κB to initiate transcription in accordance with an immune or inflammatory response. It is believed that HIV-1 protease inhibitors at least partially hinder the phosphorylation and/or proteolysis that affect this enzymatic digestion.

As used herein, “treatment” includes, but is not limited to, ameliorating a disease, lessening the severity of its complications, preventing it from manifesting, preventing it from recurring, merely preventing it from worsening, mitigating an inflammatory response included therein, or a therapeutic effort to affect any of the aforementioned, even if such therapeutic effort is ultimately unsuccessful.

Protease inhibitors are frequently used in the treatment of HIV and AIDS, yet the inhibitors that are suitable for the treatment of HIV and AIDS are not indicated for any other therapeutic purpose. The HIV-1 protease inhibitors function by preventing the cleavage of proteins that would otherwise produce infectious viral particles. Examples of these protease inhibitors may include, but are not limited to, nelfinavir, ritonavir, saquinavir, amprenavir, indinavir, lopinavir, their derivatives, analogs, equivalents, pharmaceutical or other combinations, and the like (hereinafter “protease inhibitors”). Further, protease inhibitors are generally administered in an oral dosage of from about 400 mg to about 1200 mg, depending on such variables as the particular inhibitor being administered, the age, sex, and weight of the patient, the severity of the disease condition, and the nature of the disease condition being treated.

While protease inhibitors are conventionally administered for their capacity to prevent the cleavage of various proteins, and to thereby slow or halt the progression of HIV-1 disease, protease inhibitors may also be used in the treatment of a cellular inflammatory or abnormally exaggerated immune response. In particular, both separate and apart from their capacity to hinder the progression of HIV-1 disease, HIV-1 protease inhibitors may also down-modulate immune activation and thereby function as anti-inflammatory agents.

The HIV-1 protease inhibitors of the present invention are believed to down-modulate, and not entirely block, the NF-κB cell signaling pathway. The NF-κB cell signaling pathway is important for cell survival. Entirely blocking this cell signaling pathway would likely have toxic effects, potentially manifesting as cell and tissue death and possibly immune suppression in a patient.

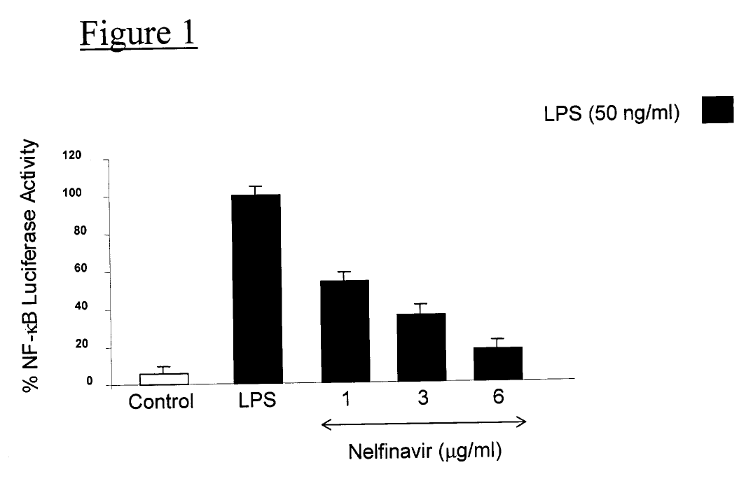

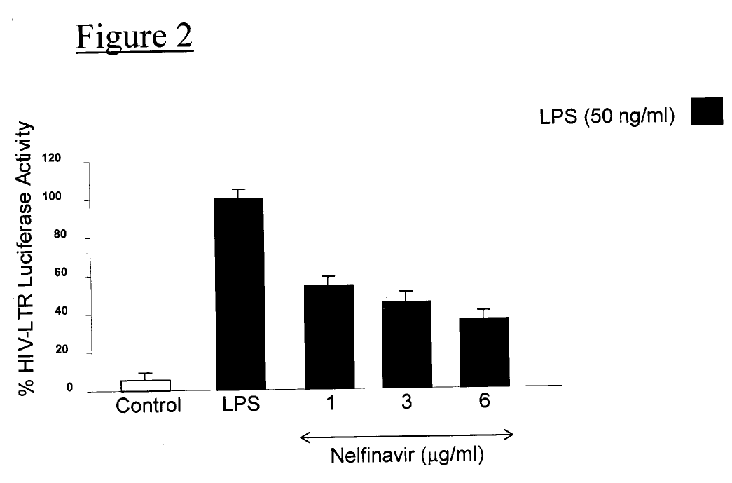

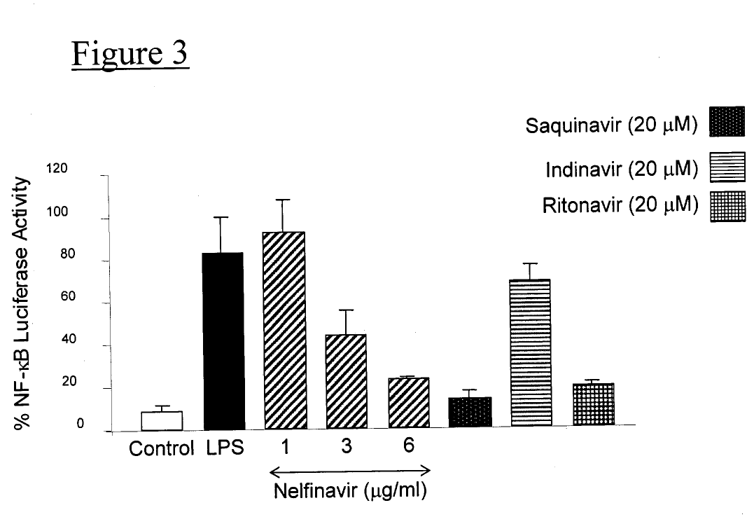

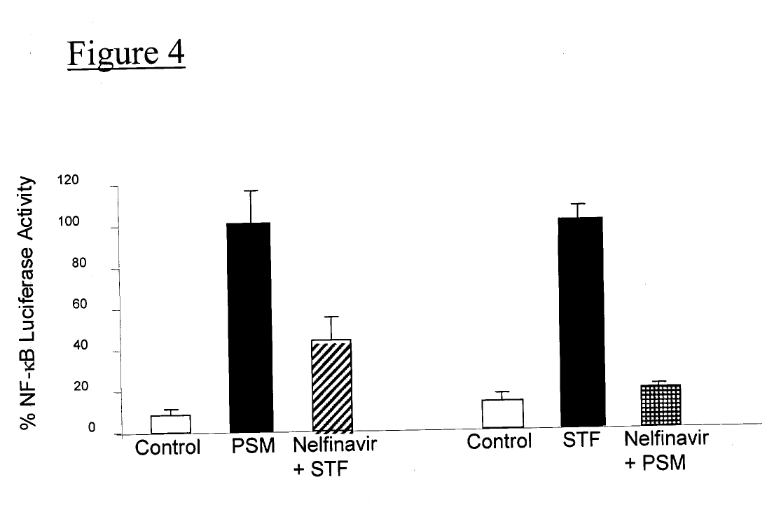

As depicted in FIG. 1, the protease inhibitors of the present invention surprisingly block activation of the NF-κB cell-signaling pathway in a dose-dependent manner. Treatment of HMEC with nelfinavir prior to cell stimulation with LPS decreased the NF-κB activity normally associated with such stimulation. Moreover, this decrease was related to the concentration of nelfinavir with which HMEC were treated prior to exposure to LPS. Similar results were obtained in examining the HIV-LTR pathway in HMEC, as seen in FIG. 2. Comparative data was obtained by treating HMEC with a variety of protease inhibitors prior to LPS exposure. As depicted in FIG. 3, HMEC were pre-treated with nelfinavir, saquinavir, indinavir, and ritonavir. All protease inhibitors blocked the NF-κB signaling pathway; saquinavir and ritonavir being more potent than indinavir, and HMEC again exhibiting a dose-dependent response to nelfinavir. While the protease inhibitors of the present invention demonstrated a capacity to block the cell signaling pathway normally triggered by gram negative microbial antigens (e.g., LPS), the protease inhibitors are also able to block antigens related to gram positive microbial antigens. As depicted in FIG. 4, the pre-treatment of HMEC with nelfinavir blocked both STF and PSM.

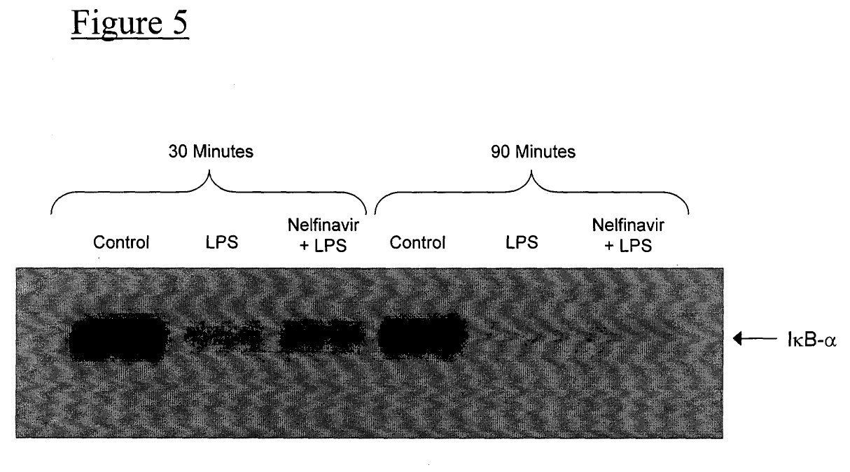

It is believed that the one way in which the protease inhibitors of the present invention may operate to inhibit the NF-κB cell signaling pathway is by inhibiting the degradation of IκB-α, an NF-κB inhibitory protein. As depicted in FIG. 5, Western blot analysis revealed that nelfinavir delayed the degradation of this protein in cells stimulated by LPS.

The protease inhibitors of the present invention may therefore be used in the treatment of a variety of illnesses wherein an inflammatory or immune response is evidenced. Examples of such illnesses or disease conditions may include, but are not limited to, sepsis and severe sepsis; various forms of arthritis, such as rheumatoid arthritis, osteoarthritis, inflammatory arthritis, psoriatic arthritis, and gout; inflammatory myocarditis; glomerulonephritis; inflammatory conditions of the gastrointestinal tract, such as inflammatory bowel disease, ulcerative colitis, and Crohn's Disease; neurologic inflammatory conditions, such as meningitis; and post infectious inflammatory conditions. The protease inhibitors of the present invention may also be used to treat a number of autoimmune diseases other than HIV and AIDS, such as, by way of example, lupus. The protease inhibitors of the present invention may thus be used to treat any disease condition where it may be advantageous to hinder the activation of cytokines, and any other disease, condition, or response where it may be advantageous to down-modulate the NF-κB cell signaling pathway.

The protease inhibitors of the present invention may be administered in any suitable form and at any suitable dosage; both of which may be readily ascertained without undue experimentation by one of sufficient skill in the art in pharmacology. The form of administration may include, for example, an oral form, such as a capsule, tablet, solution, or suspension; an intravenous form; an injectable form; an implantable form, such as a sustained release mechanism, or a biodegradable polymer unit; or any other suitable mechanism by which a therapeutic agent may be delivered to a patient. The dosage may similarly be determined in accordance with the selected form of administration. By way of example, in the treatment of HIV and AIDS, protease inhibitors are generally administered orally in an amount of from about 500 mg/day to about 3000 mg/day, with individual dosage ranging from about 400 mg to 1200 mg per administration. It will be readily appreciated by one of skill in the medical arts that dosage levels may vary widely depending on the form of administration and the particular characteristics of a patient as well as the disease condition.

In another embodiment of the present invention, a protease inhibitor may be supplemented and/or combined with another therapeutic agent to provide a therapeutic composition or treatment regimen effective in the treatment of an inflammatory or immune response, as discussed above. Additional therapeutic agents may include, but are not limited to, agents known to be effective in the treatment of arthritis, sepsis, severe sepsis, or other inflammatory conditions; antibodies effective against inflammatory cytokines, such as an anti-Tumor Necrosis Factor (TNF) antibody or an anti-lnterleukin-1 (IL1) antibody; or a cyclooxygenase (COX)-2 inhibitor.

In yet another embodiment of the present invention, an additional therapeutic agent may be provided in a form compatible with the form of the protease inhibitor (e.g., both are suitable for oral administration, and may further be combined into a singular pharmaceutical). In such an instance, a therapeutic composition may be generated by a combination of a compatible protease inhibitor and a therapeutic agent.

However, where a medicinal active and protease inhibitor are incompatible (e.g., a medicinal active is suitable for intravenous administration and a protease inhibitor is suitable for oral administration), then a protease inhibitor and a medicinal active may be administered to a patent separately, as part of a treatment regimen in accordance with yet another embodiment of the present invention. Such incompatibility may owe to the fact that particular pharmaceuticals (e.g., numerous protease inhibitors and medicinal actives) may be formulated in an intravenous form, they are better suited to oral administration. This may be due to aspects of their molecular structure, their preferred delivery route in the human body, or other factors. Yet, an example of a treatment regimen may include oral administration of a protease inhibitor in conjunction with intravenous administration of an anti-IL1 antibody.

The combination of a protease inhibitor with an additional therapeutic agent may provide more robust medical care for the treatment of inflammation or other disease conditions treated in accordance with the methods of the present invention. By way of example, a protease inhibitor may be administered to down-modulate the NF-κB pathway in conjunction with the administration of a medicinal active that targets another cell signaling pathway. Alternatively, the medicinal active may provide some other form of relief for the disease condition; one not predicated on affecting a cell signaling pathway. Therefore, the administration of a combination of these two components either in a singular pharmaceutical or via a treatment regimen may target multiple aspects of a disease condition, even if the manifestation of this condition is solely, e.g., inflammation.

In particular, down-modulation of NF-κB has known benefits in the treatment of various forms of arthritis, and may provide sufficient medicinal therapy to obviate the condition in its entirety. However, other inflammatory conditions, such as sepsis and severe sepsis, may not be fully remedied by the administration of a protease inhibitor alone, though the protease inhibitor may substantially reduce the severity of the disease condition. This may be due to the fact that a multitude of cell signaling pathways are involved in the inflammatory response associated with sepsis and severe sepsis. Thus, although a protease inhibitor may affect the NF-κB cell signaling pathway, and potentially other pathways as well, it may remain insufficient to completely treat the disease condition without additional pharmaceutical intervention. Accordingly, the combination of a protease inhibitor with a medicinal active that targets a cell signaling pathway other than the NF-κB pathway may be advantageous, particularly in the treatment of sepsis and severe sepsis.

### EXAMPLES

The Examples discussed herein demonstrate that HIV-1 protease inhibitors may be effective in the treatment of disease conditions that include a cellular inflammatory or immune response. The Examples further demonstrate that HIV-1 protease inhibitors may down-modulate the NF-κB cell signal transduction pathway, by hindering the digestion of an IκB protein/NF-κB complex into its constituent parts; thereby significantly impacting the inflammatory response.

### EXAMPLE 1

### Cells and Reagents

Immortalized human dermal endothelial cells (HMEC) were a generous gift from Dr. Candal (Centers for Disease Control and Prevention; Atlanta, Ga.). HMEC were cultured in MCDB-131 medium (available from BD Biosciences; Bedford, Mass.), supplemented with 10% heat inactivated fetal bovine serum (“FBS,” available from Gemini Bio-Products, Inc.; Woodland, Calif.), 2 mM glutamine (available from Sigma Chemicals; St Louis, Mo., hereinafter “Sigma”), and 100 μg/ml penicillin and streptomycin (available from Omega Scientific, Inc.; Tarzana, Calif.) in 24-well plates. The cells were routinely used between passages 10 and 14 as described in Faure et al., “Bacterial Lipopolysaccharide activates NF-κB through toll-like receptor 4 (TLR-4) in cultured human dermal endothelial cells. Differential expression of TLR4 and TLR-2 in endothelial cells,” J. Biol. Chem. 275(15):11058-11063 (2000).

Phenol soluble modulin (PSM), which was purified by phenol extraction of supernatants of stationary S. epidermidis, was obtained from Seymour Klebanoff (University of Wash.; Seattle, Wash.). Mehlin et al., “An inflammatory polypeptide complex from Staphylococcus epidermidis: isolation and characterization,” J. Exp. Med. 189(6):907-918 (1999).

Soluble tuberculosis factor (STF) was obtained from Terry K. Means and Matthew J. Fenton (Boston University; Boston, Mass.). All reagents were verified to be LPS free by the Limulus amebocyte lysate assay (obtained from Pyrotell, Assoc. of Cape Cod; Cape Cod, Mass.; <0.03 EU/ml). Highly purified, phenol-water extracted, and protein-free (<0.0008% protein) Escherichia coli LPS, was obtained from S. N. Vogel (Uniformed Services University; Bethesda, Md.). Hirschfeld et al., “Cutting edge: repurification of lipopolysaccharide eliminates signaling through both human and murine toll-like receptor 2,” J. Immunol. 165(2):618-622 (2000).

Nelfinavir was obtained from Agouron Pharmaceuticals (San Diego, Calif.). Other HIV-1 protease inhibitors (i.e., ritonavir, saquinavir, and indinavir) were obtained from Dr. Eric Daar (Harbor-UCLA Medical Center; Los Angeles, Calif.).

### EXAMPLE 2

### TNF-α Analysis

After five hours of LPS stimulation, supernatants from HMEC were analyzed for TNF-α production by using an ELISA kit (obtained from R&D Systems; Minneapolis, Minn.) according to manufacturer's instructions. All data for TNF-α represent the average of triplicate samples ±S.D. Each experiment was repeated at least twice.

### EXAMPLE 3

### Expression Vectors

Wild-type human TLR-2 was a gift from Ruslan Medzhitov (Yale University; New Haven, Conn.). Reporter genes pCMV-β-galactosidase (0.5 μg), ELAM-NF-κB-luciferase (0.5 μg) and interleukin (IL)-6-luciferase (0.5 μg) were used.

### EXAMPLE 4

### Transfection of Human Dermal Endothelial Cells

HMEC were plated at a concentration of 50,000 cells/well in 24-well plates and cultured in MCDB-131 with 10% serum as described above overnight. Cells were co-transfected the following day with FuGene 6 Transfection Reagent (obtained from Boehringer Mannheim; Indianapolis, Ind.) following manufacturer's instructions. Faure et al. at 11058.

The reporter genes pCMV-β-galactosidase (0.1 μg) and HIV-LTRwt-Luc (0.1 μg) expression vector (0.1 μg) were transfected into HMEC with or without hTLR2 (0.3 μg) cDNA. Cells were transfected for 24 hours and then stimulated for 6 hours with various concentrations of LPS and/or STF or PSM suspended in growth media. Cells were then lysed and luciferase activity was measured with a Promega kit (obtained from Promega; Madison, Wis.) and with a luminometer.

β-galactosidase activity was determined by colorimetric assay. Zhang et al., “Bacterial lipopolysaccharide activates nuclear factor-κB through interleukin-1 signaling mediators in cultured human dermal endothelial cells and mononuclear phagocytes,” J. Biol. Chem. 274(12):7611-7614 (1999).

### EXAMPLE 5

### 20S Proteasome Assay

The effect of nelfinavir on 20S proteasome activity was assessed using a 20S Proteasome Assay Kit (obtained from Biomol Research Laboratories, Inc.; Plymouth Meeting, Pa.) according to manufacturer's instructions. Briefly, erythrocyte 20S proteasome that is preactivated by SDS was added to Suc-LLVY-AMC fluorogenic peptide substrate, which is used to measure the chymotrypsin like peptidase activity, with or without nelfinavir. A proteasome inhibitor, lactacystin, was included as a control. The microtiter plate was read at an approximate excitation of 360 nm and at emission 460 nm. All data represent the average of triplicate samples ±S.D.

### EXAMPLE 6

### Measurement of LDH Activity

For the quantification of nelfinavir-induced cell death, lactate dehydrogenase (LDH) activity in HMEC culture supernatants was measured with a cytotoxicity detection kit (obtained from Roche Diagnostics; Indianapolis, Ind., hereinafter “Roche”) according to manufacturer's instructions. Percentage of LDH activity in the supernatants was calculated according to the following: [(Experimental Value—LDH activity released from untreated cells)/(Maximum Releasable LDH Activity in the Cells by 1% Triton X-100-LDH Activity Released from Untreated Cells)]×100.

### EXAMPLE 7

### Assessment of IκBα Degradation

Conditioned endothelial cells were lysed for 30 minutes on ice in a lysis buffer containing 0.1 mM ethylenediamine tetra-acetic acid (“EDTA,” available from Sigma), 10 mM NaF, 1 mM Na3VO4, 0.1% Triton X-100, 20 mM Tris-HCl (pH 7.5), and 1 μg/ml each of the protease inhibitors pepstatin, leupeptin, aprotinin, antipain, and chymostatin (all available from Roche).

Following lysis, cell debris was removed by centrifugation (14,000×g, 4° C., 1-2 minutes). Protein concentrations were determined using the Bradford assay. 50 μg of total protein was added in Laemmli buffer, boiled for 5 minutes, resolved by 10% sodium dodecyl sulphate polyacrylamide gel electrophoresis (“SDS-PAGE”) in Tris/glycine/SDS buffer (25 mM Tris, 250 mM glycine, 0.1% SDS), and blotted onto Immunobilon P transfer membranes (obtained from Amersham Pharmacia Biotech UK Ltd.; Buckinghamshire, England, hereinafter “Amersham”) (100 V, 1.5 hours, 4° C.). After blocking overnight in PBST (1×PBS containing 0.1% Tween 20; available from Sigma) containing 5% nonfat milk, membranes were washed three times in PBST and probed for 3 hours at 4° C. with anti-IκBα (final concentration 1 μg/ml) in PBST with 2.5% nonfat dry milk for first antibody and 1 hour for second antibody at room temperature. Membranes were thereafter and washed five times in PBST, and bands were detected using ECL reagents (obtained from Amersham) according to manufacturer's description.

### EXAMPLE 8

### HIV-1 Protease Inhibitors Block LPS-Induced NF-κB Activation in a Dose- and Time-Dependent Manner

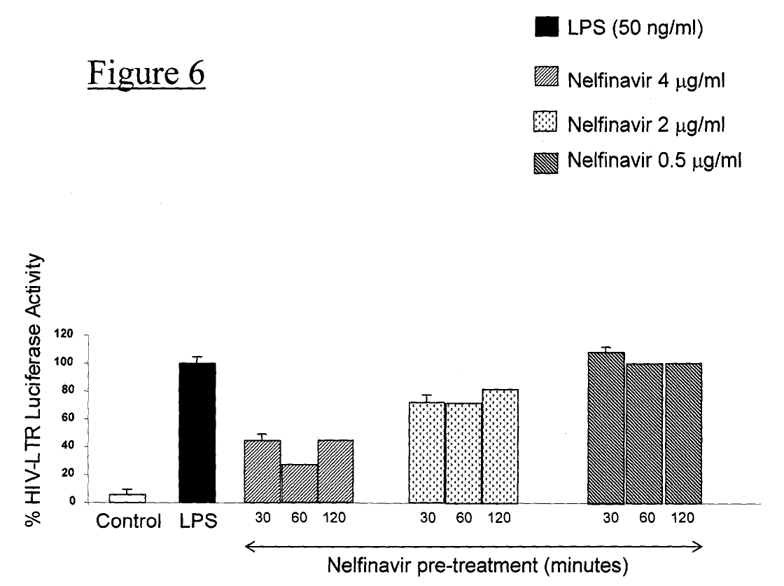

We treated HMEC with various concentrations of HIV-1 protease inhibitor (i.e., nelfinavir) before and at the time of LPS stimulation and measured luciferase activity to determine NF-κB activation. Pretreatment with nelfinavir inhibited LPS-induced NF-κB activation in both a dose-dependent (FIG. 1) and time-dependent manner (FIG. 6).

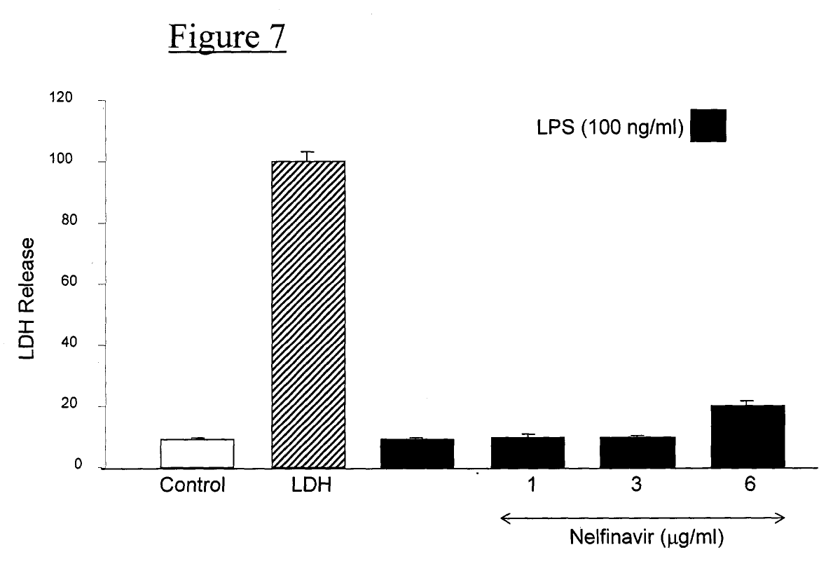

Similarly, 1 hour pretreatment of cells with ritonavir, saquinavir, and indinavir led to down-regulation of LPS-induced NF-κB activation (FIG. 3). Lactate dehydrogenase assay was performed to assess cell death, which was not induced by concentrations of nelfinavir used in the experiments (FIG. 7). These results suggest that HIV-1 protease inhibitors do not induce cell death at the concentrations tested.

### EXAMPLE 9

### Protease Inhibitor Pretreatment of HMEC Blocks TNF-α-Induced NF-κB Activation

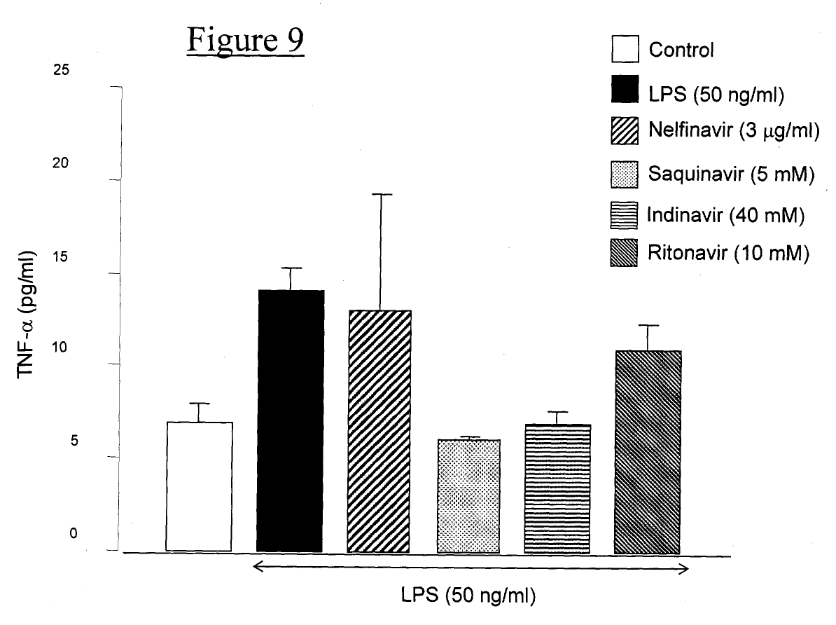

We next assessed whether protease inhibitor pretreatment of HMEC would block TNF-α-induced NF-κB activation. One-hour protease inhibitor (i.e., nelfinavir, ritonavir, saquinavir, and indinavir) pretreatment of HMEC down-regulated TNF-α (100 ng/ml) induced NF-κB activation (FIG. 9). The effect of ritonavir, saquinavir, and indinavir to block TNF-α-induced NF-κB activation was dose-dependent. Similar to their effect on LPS-induced NF-κB activation, different protease inhibitors had different potencies to inhibit TNF-α-induced NF-κB activation. Thus, it is believed that HIV-1 protease inhibitors inhibit TNF-α-induced NF-κB activation.

### EXAMPLE 10

### Protease Inhibitor Pretreatment of HMEC Blocks LPS-Induced IL-6 Transcription

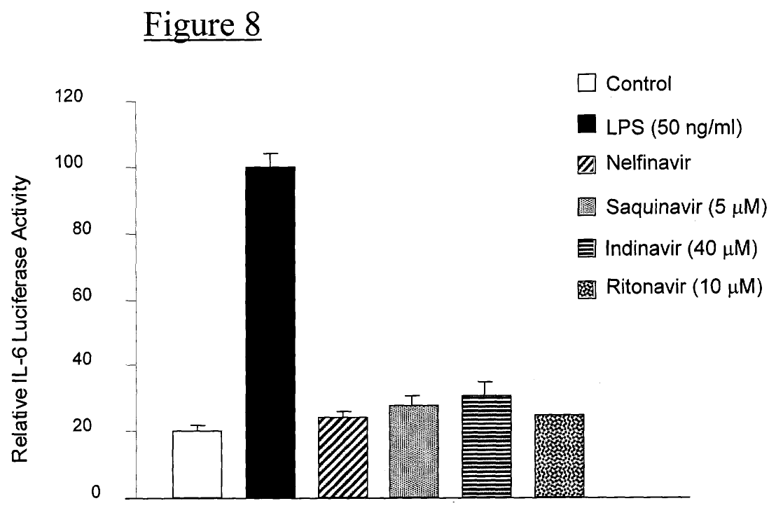

Interleukin-6 (IL-6) is a known proinflammatory cytokine that mediates development of LPS-induced sepsis and septic shock. We assessed whether HIV-1 protease inhibitor pretreatment of HMEC transiently transfected with an IL-6-promoter-luciferase construct down-regulated the LPS-induced luciferase activation. We observed that a 1-hour preteatment of HMEC with HIV-1 protease inhibitors downregulated LPS-induced luciferase activity (FIG. 8). These results suggest that protease inhibitors block LPS-induced IL-6 production.

### EXAMPLE 11

### Protease Inhibitor Pretreatment of HMEC Blocks NF-κB Activation Induced by Gram-Positive Bacteria and Mycobacteria

Besides gram-negative bacterial LPS, gram positive bacteria cell wall components such as lipoteichoic acid, peptidoglycan, phenol soluble modulin (PSM) from S. epidermidis, and mycobacterial cell wall antigens such as soluble tuberculosis factor (STF), have been shown to induce NF-κB activation and proinflammatory cytokine production. We examined whether the down-regulatory effect of HIV-1 protease inhibitors was specific to gram-negative bacterial cell wall component LPS or whether HIV-1 protease inhibitors also blocked PSM- and STF-induced NF-κB activation. Similar to LPS, one-hour pretreatment of HMEC with HIV-1 protease inhibitors blocked STF- and PSM-induced NF-κB activation (FIG. 4).

### EXAMPLE 12

### Nelfinavir Pretreatment of HMEC Delays LPS-Induced IκB-α Degradation

Upon exposure of immune system cells to bacterial and mycobacterial antigens, IκB-α degradation is the key step before NF-κB activation. We assessed the effect of nelfinavir pretreatment of HMEC on LPS-induced IκB-α degradation. HMEC were pretreated with nelfinavir for one hour and stimulated with LPS for 30, 60, and 90 minutes. IκB-α degradation was assessed by Western Blot analysis for phosphorylated IκB-α. As expected, LPS stimulation led to degradation of phosphorylated IκB-α in HMEC; whereas in nelfinavir pretreated HMEC there was a delay in LPS-induced IκB-α degradation (FIG. 5).

### EXAMPLE 13

### Nelfinavir does not Inhibit Chymotrypsin-Like Activity of 20S Proteasome

Ritonavir has been shown to inhibit the chymotrypsin-like activity of 20s proteasome; an element that mediates LPS-induced IκB-α degradation. We assessed whether the ability of nelfinavir to delay LPS-induced IκB-α degradation was due to an inhibition of the chymotrypsin-like activity of 20s proteasome. Nelfinavir did not inhibit the chymotrypsin-like activity of 20s proteasome; rather the inhibition was due to dimethyl sulfoxide (DMSO), which is used to dissolve nelfinavir. These results suggest that inhibitory effect of nelfinavir on NF-κB activation is not mediated through inhibition of the chymotrypsin-like activity of 20s proteasome.

While the description above refers to particular embodiments of the present invention, it will be understood that many modifications may be made without departing from the spirit thereof. For instance, the protease inhibitors of the present invention may be used in the treatment of any number of conditions where inflammation is observed, as would be readily recognized by one skilled in the art and without undue experimentation. The accompanying claims are intended to cover such modifications as would fall within the true scope and spirit of the present invention.

The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

We claim:

1. A method of mitigating an inflammatory response in a patient, the method comprising the step of administering an HIV-protease inhibitor to the patient in an amount effective to mitigate the inflammatory response.

2. The method of claim 1, wherein the HIV-protease inhibitor is selected from the group consisting of nelfinavir, ritonavir, saquinavir, amprenavir, indinavir, lopinavir, a derivative of any of the foregoing protease inhibitors, an analog of any of the foregoing protease inhibitors, a pharmaceutical equivalent of any of the foregoing protease inhibitors, and a combination of any of the foregoing protease inhibitors.

3. The method of claim 1, wherein the amount is from about 500 mg/day to about 3,000 mg/day.

4. The method of claim 1, the method further comprising the step of administering an additional therapeutic agent in an amount effective to further mitigate the inflammatory response.

5. The method of claim 4, wherein the therapeutic agent is selected from the group consisting of an anti-TNF antibody, an anti-IL-1 antibody, and a COX-2 inhibitor.

6. The method of claim 4, the method further comprising the step of combining the therapeutic agent with the protease inhibitor in a pharmaceutically effective carrier.

7. The method of claim 4, the method further comprising the step of administering the therapeutic agent separately from the protease inhibitor in a treatment regimen.

8. A method of treating a disease condition that includes an inflammatory response in a patient, the method comprising the step of administering an HIV-protease inhibitor to the patient in an amount effective to mitigate the inflammatory response.

9. The method of claim 8, wherein the disease condition is selected from the group consisting of sepsis, severe sepsis, arthritis, rheumatoid arthritis, osteoarthritis, inflammatory arthritis, psoriatic arthritis, gout, an inflammatory condition of the gastrointestinal tract, inflammatory bowel disease, ulcerative colitis, Crohn's Disease, a neurologic inflammatory condition, meningitis, inflammatory myocarditis, glomerulonephritis, an autoimmune disease, and lupus.

10. The method of claim 8, wherein the HIV-protease inhibitor is selected from the group consisting of nelfinavir, ritonavir, saquinavir, amprenavir, indinavir, lopinavir, a derivative of any of the foregoing protease inhibitors, an analog of any of the foregoing protease inhibitors, a pharmaceutical equivalent of any of the foregoing protease inhibitors, and a combination of any of the foregoing protease inhibitors.

11. The method of claim 8, wherein the amount is from about 500 mg/day to about 3,000 mg/day.

12. The method of claim 8, the method further comprising the step of administering an additional therapeutic agent in an amount effective to further mitigate the inflammatory response.

13. The method of claim 12, wherein the therapeutic agent is selected from the group consisting of an anti-TNF antibody, an anti-IL-1 antibody, and a COX-2 inhibitor.

14. The method of claim 12, the method further comprising the step of combining the therapeutic agent with the protease inhibitor in a pharmaceutically effective carrier.

15. The method of claim 12, the method further comprising the step of administering the therapeutic agent separately from the protease inhibitor in a treatment regimen.

16. A method of treating a disease condition in a patient in which it is advantageous to hinder NF-κB cell signal transduction, the method comprising the step of administering an HIV-protease inhibitor to the patient in an amount effective to treat the disease condition.

17. The method of claim 16, wherein the disease condition is selected from the group consisting of sepsis, severe sepsis, arthritis, rheumatoid arthritis, osteoarthritis, inflammatory arthritis, psoriatic arthritis, gout, an inflammatory condition of the gastrointestinal tract, inflammatory bowel disease, ulcerative colitis, Crohn's Disease, a neurologic inflammatory condition, meningitis, an autoimmune disease, and lupus.

18. The method of claim 18, wherein the HIV-protease inhibitor is selected from the group consisting of nelfinavir, ritonavir, saquinavir, amprenavir, indinavir, lopinavir, a derivative of any of the foregoing protease inhibitors, an analog of any of the foregoing protease inhibitors, a pharmaceutical equivalent of any of the foregoing protease inhibitors, and a combination of any of the foregoing protease inhibitors.

19. The method of claim 16, wherein said amount is from about 500 mg/day to about 3,000 mg/day.

20. The method of claim 16, the method further comprising the step of administering an additional therapeutic agent in an amount effective to further mitigate the inflammatory response.

21. The method of claim 20, wherein the therapeutic agent is selected from the group consisting of an anti-TNF antibody, an anti-IL-1 antibody, and a COX-2 inhibitor.

22. The method of claim 20, the method further comprising the step of combining the therapeutic agent with the protease inhibitor in a pharmaceutically effective carrier.

23. The method of claim 20, the method further comprising the step of administering the therapeutic agent separately from the protease inhibitor in a treatment regimen.
